(19) United States
(12) Patent Application Publication
(10) Pub. No.: US 2024/0255532 A1
(43) Pub. Date: Aug. 1, 2024

(54) Biomarkers for the Detection of Thrombosis or Coagulation-Related Disorders and Their Applications

(71) Applicant: YIGONG RUISI FUJIAN ENGINEERING RESCENTER CO LTD., Fuzhou City, Fujian Province (CN)

(72) Inventor: Zhe Lin, Fuzhou City, Fujian Province (CN)

(21) Appl. No.: 18/560,680

(22) PCT Filed: May 13, 2022
(86) PCT No.: PCT/CN2022/092581

§ 371 Date: Jan. 11, 2024

(30) Foreign Application Priority Data
May 14, 2021 (CN) 202110526415.2

(51) Int. Cl. G01N 33/86; G01N 33/68

(52) U.S. Cl. CPC G01N 33/86; G01N 33/6893; G01N 2333/91085; G01N 2800/224; G01N 2800/226; G01N 2800/2871

(57) ABSTRACT

The present invention relates to the technical field of thrombosis and hemostasis-related disease detection technologies, and in particular to a biomarker and an application thereof in thrombotic and coagulation-related disorders detection. The biomarker comprises an activated peptide of Coagulation Factor XIII (FXIIIAP) and/or an A subunit of Coagulation Factor XIII (FXIIIA). The diagnostic kit provided by the present invention comprises a capture antibody and a detection antibody, which is a specific antibody prepared by using FXIIIAP as an antigen. The biomarker of the present invention has a promising application prospect of rapid screening and diagnosis of thrombotic and coagulation-related diseases, including strokes, can significantly improve the sensitivity and specificity of existing diagnostic methods, speculate the thrombus formation timing and extent, and can predict the severity and prognosis of the disease.

### FIELD OF THE INVENTION

The present invention relates to the technological field of thrombosis or coagulation-related disorder detection, and in particular to a biomarker for detecting thrombosis or coagulation-related disorders and an application thereof.

### BACKGROUND OF THE INVENTION

Thrombus formation and associated coagulation disorders are pivotal diagnostic elements in clinical settings, encompassing a wide spectrum of diseases such as Cerebral Venous Thrombosis (CVT), Renal Vein Thrombosis (RVT), Venous Thromboembolism (VTE), and Myocardial Infarction (MI). Particularly, the early diagnosis and treatment of conditions like cerebral venous thrombosis and myocardial infarction are of significant importance. For instance, in the context of cerebral venous thrombosis, a major cause of ischemic stroke, established diagnostic methods like Computed Tomography (CT) and Magnetic Resonance Imaging (MRI) are notably effective in determining the incidence, type, and severity of strokes. However, these modalities are not only costly and time-consuming but also lack efficiency in early detection, often resulting in missed opportunities for optimal treatment. In contrast, CAT scans (Computerized Axial Tomography) are better suited for diagnosing hemorrhagic strokes. Despite the relative sensitivity in differentiating hemorrhagic stroke thereof, CAT scans are less sensitive to cerebral ischemia during stroke assessments, usually becoming positive 24-36 hours after stroke, with approximately 50% of ischemic strokes being undetectable via CAT scans. Consequently, by the time existing diagnostic technologies confirm a stroke, the window for early intervention is often lost.

For diagnosing acute ischemic and hemorrhagic strokes, the prevalent clinical methods lack sufficient sensitivity, accuracy, and timeliness, leading to potential misdiagnoses or missed diagnoses, thereby delaying the treatment.

Recent researches indicate that employing molecular biomarkers for rapid screening and differential diagnosis of stroke types offers a novel approach to address these challenges.

Biomarkers refer to biochemical indicators that can mark changes in the structure or function of an organism's organs, tissues or cells. Therefore, the detection of biomarkers can be used for disease diagnosis and provide a scientific basis for subsequent treatment options.

Various stroke biomarkers are already known. Molecular biomarkers related to stroke have been disclosed in the invention patent with publication number CN103299191A. Methods for diagnosing and differentiating strokes are disclosed in the invention patent with publication number CN1339108A. Thrombus formation serves as a crucial diagnostic indicator for cardiovascular diseases like ischemic strokes. The current clinical screening for thrombi predominantly involves D-dimer test, primarily utilized in diagnosing Venous Thromboembolism (VTE), Deep Vein Thrombosis (DVT), and Pulmonary Embolism (PE). D-dimer testing has shown promise in many instances. However, due to various influencing factors and intrinsic limitations of D-dimers themselves, there are still many controversies in the clinical use of D-dimer test. Primarily, D-dimers originate from plasmin-dissolved crosslinked fibrin clots, reflecting the status of fibrinolysis (thrombolysis) rather than direct thrombus formation. All factors affecting thrombolysis can influence D-dimer test outcomes. For example, for the patients with fibrinolytic disorders, those on oral anticoagulants, or those with VTE symptoms persisting for over 14 days, it may yield false-negative results. Moreover, D-dimers, indicative of the thrombus dissolution process and having a prolonged half-life (13-23 hours), can produce false-positive results when other coagulation triggers are present, like inflammation or pregnancy. D-dimers also exhibit a high rate of missed diagnosis in clinical tests. For instance, there's a 20% missed diagnosis rate for CVT, higher in early stages (symptoms appearing in less than 24 hours) at about 50%. For general venous thrombosis, D-dimer diagnosis also shows a 50% missed diagnosis rate. Particularly, for early-stage newly formed thrombi, it can give false-negative results.

Due to the extended half-life of D-dimers, they fail to accurately and timely reflect the occurrence of bleeding and coagulation when there is existing coagulation disorders. Moreover, the ambiguous definition of the D-dimer analyte, along with varying detection methodologies based on monoclonal antibodies that recognize different fibrin fragments or surface structures, result in the absence of international standard reference preparations or calibrators. Coupled with the low sensitivity and specificity thereof, the application of D-dimers is still controversial. Additionally, the detection results lack therapeutic guidance, failing to accurately and promptly reflect the body's coagulation state, thereby not providing indications for treatment strategies, such as the effectiveness of anticoagulant therapies, or confirming the presence of new thrombi, since old thrombi can still be dissolved to produce D-dimers.

### SUMMARY OF THE INVENTION

### Technical Problems to be Solved

Recognizing the deficiencies of existing technologies, the present invention introduces a biomarker for directly detecting thrombus formation, distinct from existing biomarkers that indirectly detect tissue stress responses to stroke. The biomarker, either used alone or in combination with D-dimer test, offers high sensitivity and accuracy in detecting and screening thrombosis or coagulation-related diseases.

More particularly, the invention provides a biomarker applicable for detecting thrombosis or coagulation-related diseases.

Additionally, the invention includes an application of said biomarker in products designed for detecting thrombosis or coagulation-related diseases and for evaluating treatment efficacy.

Further, the invention exhibits a reagent kit tailored for detecting thrombosis or coagulation-related diseases.

### Technical Solutions

To achieve the aforementioned objectives, the invention the following principal technical solutions:

In a first aspect, the invention provides a biomarker for detecting thrombosis or coagulation-related diseases, comprising FXIIIAP or FXIIIA, wherein FXIIIAP is an activation peptide of Coagulation Factor XIII, and FXIIIA is an A subunit of Coagulation Factor XIII.

Optionally, the biomarker may include a combination of FXIIIAP and D-dimer.

Optionally, the relevant coagulation disorders include conditions like stroke, cerebral venous thrombosis, renal vein thrombosis, venous thrombosis, or myocardial infarction.

In a second aspect, the invention also introduces a diagnostic method for thrombosis or cardiovascular diseases, involving:
S1: Generating specific antibodies using either complete or partial sequence of FXIIIAP as an antigen.
S2: Forming a complex with free FXIIIAP in test samples using the derived specific antibodies.
S3: Determining FXIII activation and diagnosing thrombosis or cardiovascular-related diseases by detecting the complex.

Optionally, the test sample can be blood or plasma.

Optionally, specific antibodies are generated using a partial sequence of FXIIIAP, including sequences SETSRTAFGGRRAVPPNNSNAAEDDLPTVELQGVV or RTAFGGRRAVPPNN.

In a third aspect, the invention provides an application of the said biomarker in products for detection and treatment effect assessment of thrombosis or coagulation-related diseases.

Optionally, the products for detection and treatment effect assessment of thrombosis or coagulation-related diseases may include a reagent kit or a reagent.

In a fourth aspect, the invention offers a reagent kit for detecting thrombosis or coagulation-related diseases, wherein the kit's capture antibodies and/or detection antibodies are specific antibodies produced using the biomarker as an antigen.

Optionally, the reagent kit can be a double-antibody sandwich ELISA detection kit or a dry-type immunofluorescence detection kit, comprising a capture antibody and a detection antibody.

Optionally, the antibodies in the kit are produced using complete or partial sequence of FXIIIAP an antigen.

Optionally, in the reagent kit for detecting thrombosis or coagulation-related disorders, the partial sequence includes the following sequence or a fragment comprising the following sequence: SETSRTAFGGRRAVPPNNSNAAEDDLPTVELQGVV or RTAFGGRRAVPPNN.

Optionally, the capture antibodies and the detection antibodies are derived from animals immunized with FXIIIAP.

Optionally, the antibodies of the invention are not limited to ELISA (Enzyme-Linked Immunosorbent Assay) but are also applicable in Colloidal Gold Method, Radioimmunoassay (RIA), Chemiluminescent Immunoassay (CLIA), Electro-Chemiluminescence Immunoassay (ECLI), Fluorescent Immunoassay like Dry-Type Immunofluorescence Method, Time-Resolved Fluoroimmunoassay (TRFIA) for detecting the aforementioned biomarkers.

### Beneficial Effects

The invention's beneficial effects include:

The invention provides a biomarker for detecting thrombosis or coagulation-related diseases, which, in combination with the traditional D-dimer biomarker, can significantly enhance sensitivity and detection rates over current technologies.

The biomarker FXIIIAP, as indicated in the invention, demonstrates superior detection performance for acute thrombosis (less than 48 hours) compared to existing technologies, specifically in contrast to D-dimer. However, due to rapid degradation of FXIIIAP 48 hours after symptom onset, it can't be detected, resulting in a very high missed diagnosis rate. Therefore, the present invention proposes combining FXIIIAP with D-dimer to effectively and substantially lower the missed diagnosis rate throughout the disease cycle, including early and late stages.

Wherein,

1. The biomarker proposed in the invention does not correlate with the fibrinolysis process, allowing detection in patients with insufficient fibrinolysis.

2. The biomarker proposed in the invention provides a more accurate reflection of new thrombus formation inside the body compared to D-dimer and is thus applicable in evaluating treatment effects and disease severity.

3. The biomarker proposed in the invention facilitates rapid diagnosis of coagulation diseases including stroke, enhances existing diagnostic sensitivity and specificity, enables inference on thrombus formation timing and extent, and predicts disease severity and prognosis.

4. The application include in the invention provides information on treatment strategies, such as guidance for anticoagulant or thrombolytic therapy, and effectively monitor the effectiveness of anticoagulant drugs.

5. The detection kits proposed in the invention are suitable for finger blood testing and long-term monitoring, possessing a significant commercial value.

6. The detection kits proposed in the invention fills a market gap for FXIIIAP diagnostic kits.

### BRIEF DESCRIPTION OF THE DRAWINGS

FIG. 1: Detection results of ischemic stroke patient samples using traditional biomarker D-dimer as a control in the invention.

FIG. 2: Detection results for the biomarker FXIIIAP in ischemic stroke patient samples as described in the invention.

FIG. 3: Sensitivity comparison of detection results for the biomarker FXIIIAP and traditional biomarker D-dimer in early ischemic stroke patient samples as described in the invention.

FIG. 4: Sensitivity graph for detection results of the biomarker combination of FXIIIAP and D-dimer in early ischemic stroke patient samples as proposed in the invention.

FIG. 5: Sensitivity comparison graph for antibodies described in Embodiment 3-1-1 (original antibody, denoted as 1-37), Embodiment 3-1-2 (improved antibody 1, denoted as 1-35), and Embodiment 3-1-4 (improved antibody 2, denoted as 5-18) used in ELISA for FXIIIAP detection.

FIG. 6: Specificity comparison graph for antibodies described in Embodiment 3-1-1 (original antibody, denoted as 1-37), Embodiment 3-1-2 (improved antibody 1, denoted as 1-35), and Embodiment 3-1-4 (improved antibody 2, denoted as 5-18) used in ELISA for FXIIIAP detection.

FIG. 7: Basic information of Patient A.

FIG. 8: The first set of thrombosis monitoring data for Patient A.

FIG. 9: The second set of thrombosis monitoring data for Patient A.

FIG. 10: Graphical representation of variations in white blood cell count and proportion of promyelocytes in Patient A.

FIG. 11: Graph of variations in Factor XIII Activation Peptide (FXIIIAP) and fibrinogen levels in Patient A.

FIG. 12: Chart depicting changes in D-dimer concentration and fibrin monomer presence in Patient A.

FIG. 13: Basic information of Patient B.

FIG. 14: The first set of thrombosis monitoring data for Patient B.

FIG. 15: The second set of thrombosis monitoring data for Patient B.

FIG. 16: Graph indicating changes in white blood cell count and proportion of promyelocyte in Patient B.

FIG. 17: Graphical representation of variations in FXIIIAP and fibrinogen levels in Patient B.

FIG. 18: Chart depicting alterations in D-dimer and fibrin monomer levels in Patient B.

FIG. 19: Basic information of Patient C.

FIG. 20: Thrombosis monitoring data for Patient C.

FIG. 21: Basic information of Patient D.

FIG. 22: Thrombosis monitoring data for Patient D.

### DETAILED DESCRIPTION OF PREFERRED EMBODIMENTS

To better explain and understand the present invention, the following detailed description is provided in conjunction with accompanying drawings:

### Embodiment Series 1

### Embodiment 1-1

A biomarker for detecting thrombosis or coagulation-related diseases: FXIIIAP (coagulation factor XIII activation peptide).

### Embodiment 1-2

A biomarker for detecting thrombosis or coagulation-related diseases: FXIIIA (A subunit of coagulation factor XIII).

### Embodiment 1-3

A biomarker for detecting thrombosis or coagulation-related diseases: a combination of FXIIIAP and FXIIIA.

### Embodiment 1-4

A biomarker for detecting thrombosis or coagulation-related diseases: a combination of FXIIIAP and D-dimer.

### Embodiment 1-5

A biomarker for detecting thrombosis or coagulation-related diseases: a combination of FXIIIA and D-dimer.

### Embodiment 1-6

A biomarker for detecting thrombosis or coagulation-related diseases: a combination of FXIIIAP, FXIIIA, and D-dimer.

In the disclosed embodiments 1-4, 1-5, and 1-6, the biomarker combinations comprising FXIIIAP or FXIIIA and D-dimer exhibited enhanced accuracy for detecting thrombotic or coagulation-related disorders across various stages. Distinct from D-dimer, the presence of FXIIIAP was independent of the fibrinolytic process, thereby enabling the application thereof in patients exhibiting suboptimal fibrinolysis. Moreover, FXIIIAP appeared earlier than D-dimer. Clinical evaluations had demonstrated superior efficacy of FXIIIAP in identifying acute-phase thrombosis (less than 2 days after onset). While D-dimer levels began to increase from the second day after symptom onset, and increased significantly on the third day. FXIIIAP levels were already elevated (15.8 ng/ml) on the first day when D-dimer levels remained comparatively lower (<500 ng/mL). This elevation in healthy individuals was typically around 4 ng/mL. On the second day, FXIIIAP concentrations notably decreased to 7.2 ng/ml and returned to the normal on the third day, illustrating a complementary relationship between D-dimer and FXIIIAP.

Studies indicated rapid in vivo degradation of FXIIIAP, suggesting that the elevated levels thereof reflected the immediate thrombotic state in the body. Compared to D-dimer, FXIIIAP more effectively reflected the formation of new thrombi, thus aiding in evaluating treatment efficacy and disease severity. However, due to the expedited degradation thereof, FXIIIAP might not be detectable in patients with advanced symptoms, resulting in a certain misdiagnosis rate. Therefore, it was necessary to combine with D-dimer to reduce the risk of missed diagnosis.

FXIIIAP is not only effective for stroke caused by thrombosis, but theoretically it will also have considerable effects on hemorrhagic stroke. When intracranial hemorrhage occurs, the permeability of the blood-brain barrier increases, and coagulation factors can pass through the blood-brain barrier. Given the very low molecular weight thereof (merely 3.91 KD), FXIIIAP readily traverses the blood-brain barrier. The brain, abundant in tissue factors, releases these upon blood-brain barrier compromise, which induce systemic coagulation. While D-dimer has been utilized to detect intracranial hemorrhage, outcomes have been suboptimal. Presently, no experimental assays employing FXIIIAP for hemorrhagic stroke diagnosis exist. Theoretically, it is possible that FXIIIAP may maintain elevated levels without reduction over time, alongside diminished FXIIIA and elevated D-dimer concentrations (high AP level, low FXIIIA level, and high D-dimer level).

The present invention comprises coagulation-related conditions, including but not limited to stroke, cerebral venous thrombosis, renal vein thrombosis, venous thrombosis, and myocardial infarction, thereby covering a spectrum of thrombosis-induced disease.

### Embodiment Series 2

### Embodiment 2-1

This embodiment relates to an application of the biomarkers as described in Embodiment 1-1 in detection products and therapeutic efficacy evaluation products for thrombosis or coagulation related diseases.

Further, in certain embodiments, the products comprise reagent kits or reagents.

### Embodiment 2-2

This embodiment relates to an application of the biomarkers as described in Embodiment 1-2 in detection products and therapeutic efficacy evaluation products for thrombosis or coagulation related diseases.

Further, in some embodiments, the products comprise reagent kits or reagents.

### Embodiment 2-3

This embodiment relates to an application of the biomarkers as described in Embodiment 1-3 in detection products and therapeutic efficacy evaluation products for thrombosis or coagulation related diseases.

Further, in some embodiments, the products comprise reagent kits or reagents.

### Embodiment 2-4

This embodiment relates to an application of the biomarkers as described in Embodiment 1-4 in detection products and therapeutic efficacy evaluation products for thrombosis or coagulation related diseases.

Further, in some embodiments, the products comprise reagent kits or reagents.

### Embodiment 2-5

This embodiment relates to an application of the biomarkers as described in Embodiment 1-5 in detection products and therapeutic efficacy evaluation products for thrombosis or coagulation related diseases.

Further, in some embodiments, the products comprise reagent kits or reagents.

### Embodiment 2-6

This embodiment relates to an application of the biomarkers as described in Embodiment 1-6 in detection products and therapeutic efficacy evaluation products for thrombosis or coagulation related diseases.

Further, in some embodiments, the products comprise reagent kits or reagents.

### Embodiment Series 3

### Embodiment 3-1

A reagent kit for detecting thrombotic or coagulation-related disorders, comprising a capture antibody and/or a detection antibody, wherein the antibodies are specific antibodies generated using the biomarkers as described in Embodiment 1-1 as antigens. In some embodiments, the kit is specifically formulated to include a dual-antibody sandwich ELISA kit comprising both capture and detection antibodies.

### Embodiment 3-1-1

On the basis of Embodiment 3-1, more specifically, the capture and detection antibodies are produced using the complete sequence of FXIIIAP as the antigen.

### Embodiment 3-1-2

On the basis of Embodiment 3-1, more specifically, the capture and detection antibodies are created using a partial sequence of FXIIIAP as the antigen, and the is partial sequence SETSRTAFGGRRAVPPNNSNAAEDDLPTVELQGVV.

### Embodiment 3-1-3

On the basis of Embodiment 3-1, more specifically, the capture and detection antibodies are created using a partial sequence of FXIIIAP as the antigen, and the partial sequence is a partial segment of SETSRTAFGGRRAVPPNNSNAAEDDLPTVELQGVV.

### Embodiment 3-1-4

On the basis of Embodiment 3-1, more specifically, the capture and detection antibodies are derived using a partial sequence of FXIIIAP as the antigen, and the partial sequence is RTAFGGRRAVPPNN.

### Embodiment 3-1-5

On the basis of Embodiment 3-1, more specifically, the capture and detection antibodies are specifically engineered using a fragment of the FXIIIAP sequence as the antigen, and the partial sequence is a partial segment of RTAFGGRRAVPPNN.

### Embodiment 3-2

A reagent kit for detecting thrombotic or coagulation-related disorders, comprising a capture antibody and/or a detection antibody, wherein the antibodies are specific antibodies generated using the biomarkers as described in Embodiment 1-2 as antigens. In some embodiments, the kit is specifically formulated to include a dual-antibody sandwich ELISA kit comprising both capture and detection antibodies.

### Embodiment 3-3

A reagent kit for detecting thrombotic or coagulation-related disorders, comprising a capture antibody and/or a detection antibody, wherein the antibodies are specific antibodies generated using the biomarkers as described in Embodiment 1-3 as antigens. In some embodiments, the kit is specifically formulated to include a dual-antibody sandwich ELISA kit comprising both capture and detection antibodies.

### Embodiment 3-4

A reagent kit for detecting thrombotic or coagulation-related disorders, comprising a capture antibody and/or a detection antibody, wherein the antibodies are specific antibodies generated using the biomarkers as described in Embodiment 1-4 as antigens. In some embodiments, the kit is specifically formulated to include a dual-antibody sandwich ELISA kit comprising both capture and detection antibodies.

Further, in some embodiments, the antibodies are sourced from animals immunized with FXIIIAP, more accurately, extracting IgG from such immunized test animals.

### Embodiment 3-5

A reagent kit for detecting thrombotic or coagulation-related disorders, comprising a capture antibody and/or a detection antibody, wherein the antibodies are specific antibodies generated using the biomarkers as described in Embodiment 1-5 as antigens. In some embodiments, the kit is specifically formulated to include a dual-antibody sandwich ELISA kit comprising both capture and detection antibodies. In some embodiments, the kit is specifically formulated to include a dual-antibody sandwich ELISA kit comprising both capture and detection antibodies.

Further, in some embodiments, the antibodies are sourced from animals immunized with FXIIIAP, more accurately, extracting IgG from such immunized test animals.

### Embodiment 3-6

A reagent kit for detecting thrombotic or coagulation-related disorders, comprising a capture antibody and/or a detection antibody, wherein the antibodies are specific antibodies generated using the biomarkers as described in Embodiment 1-6 as antigens. In some embodiments, the kit is specifically formulated to include a dual-antibody sandwich ELISA kit comprising both capture and detection antibodies. In some embodiments, the kit is specifically formulated to include a dual-antibody sandwich ELISA kit comprising both capture and detection antibodies.

Further, in some embodiments, the antibodies are sourced from animals immunized with FXIIIAP, more accurately, extracting IgG from such immunized test animals.

### Embodiment 3-7

A reagent kit for detecting thrombotic or coagulation-related disorders, comprising a capture antibody and/or a detection antibody, wherein the antibodies are specific antibodies generated using the biomarkers as described in Embodiment 1-7 as antigens. In some embodiments, the kit is specifically formulated to include a dual-antibody sandwich ELISA kit comprising both capture and detection antibodies. In some embodiments, the kit is specifically formulated to include a dual-antibody sandwich ELISA kit comprising both capture and detection antibodies.

Further, in some embodiments, the antibodies are sourced from animals immunized with FXIIIAP, more accurately, extracting IgG from such immunized test animals.

Different from D-dimer, Coagulation Factor XIII (FXIII) is the last coagulation factor activated in coagulation process, primarily responsible for cross-linking fibrin clots to stabilize thrombi. The activation thereof reflects the completion of the coagulation process, indicating formation of a stable thrombus. During the activation thereof, each FXIII molecule releases two Factor XIII Activation Peptides (FXIIIAPs). FXIIIAP, a peptide consisting of only 37 amino acids (approximately 3.91 KD), stabilizes the dimeric structure of FXIIIA₂. Upon activation by Thrombin, FXIIIAP disassociates from FXIII and enters the bloodstream as free FXIIIAP. The structural conformation of free FXIIIAP markedly differs from that bound to FXIII. The specific antibodies, produced in the present invention using synthetic FXIIIAP as an antigen, do not bind to FXIIIAP in unactivated FXIII (i.e., bound FXIIIAP). The specific antibodies in the present invention can effectively detect free FXIIIAP without binding to unactivated FXIII. This feature ensures diagnostic specificity and can accurately detect activated FXIII. Clinical experiments have demonstrated that released FXIIIAP can be detected by using ELISA or fluorescent immunological methods.

For the reagent kit proposed in the present invention, core component thereof is FXIIIAP specific antibodies produced using an improved FXIIIAP amino acid sequence. According to computer-simulated experiments: FXIIIAP, when cleaved by Thrombin and released into the bloodstream, forms a double β-strands structure with an amino acid sequence of 1-35, whereas amino acids 36 and 37 do not contribute to this structure formation. On the contrary, the two amino acids' instability directly causes variability in the carboxyl end of synthetic FXIIIAP, and the produced antibodies cannot efficiently detect native FXIIIAP. The present invention utilizes synthetic FXIIIAP with a more stable structure as an antigen (amino acid sequence 1-35), more specifically, Using the sequence SETSRTAFGGRRAVPPNNSNAAEDDLPTVELQGVV to generate specific antibodies against the stable double β-strands structure, effectively enhancing antibody sensitivity.

More particularly, upon further analysis of FXIIIAP's structure, the inventors found amino acid sequence 5-18 to be critical for the binding of FXIIIAP to FXIIIA subunit and antigenically favorable for antibody production. In the bound state with FXIIIA, the antibody binding site is masked. Once FXIIIAP becomes free, the antibody binding site is exposed. Therefore, a peptide containing the amino acid sequence 5-18 (RTAFGGRRAVPPNN) can be used for antibody production.

Specific antibodies produced using this method, relative to antibodies generated using the complete sequence of FXIIIAP (amino acid sequence 1-37, monoclonal antibody), can significantly improve sensitivity to native FXIIIAP. Enhanced sensitivity is observed in vitro tests using normal plasma; more preferably, cysteine is added at the carboxyl or amino end of the above partial sequence for binding carrier proteins. Sensitivity experiments with the improved antibodies show that antibodies produced using this method can detect more than 80% of native FXIIIAP. Additionally, as the standard sequence of FXIIIAP was used in antibody preparation, this diagnostic reagent is easier to standardize and promote compared to D-dimer.

Currently, there are no FXIIIAP detection kits on the market. The FXIIIAP detection kit provided by the present invention, utilizing specific antibodies generated using FXIIIAP as an antigen, can effectively bind free FXIIIAP in plasma without binding to FXIIIAP in unactivated FXIII (bound FXIIIAP), thus effectively detecting free FXIIIAP in plasma. The FXIIIAP detection kit can accurately detect activated FXIII for diagnosing of thrombosis or cardiovascular diseases. The kit proposed in the present invention has been clinically proven to detect released FXIIIAP using ELISA or fluorescent immunological methods.

For ease of antibody production, the standard complete sequence of FXIIIAP has cysteine added at the amino terminus to bind to the carrier protein, increasing the immunogenicity of the synthesized FXIIIAP. As the standard complete sequence of FXIIIAP (SETSRTAFGGRRAVPPNNSNAAEDDLPTVELQGVVPR, PDB Entry 1F13) is used in the preparation of specific antibodies, standardization is facilitated, making it easier to promote compared to D-dimer.

Further, in the specific antibody preparation process, a partial sequence of the synthetic FXIIIAP with more stable structure (amino acid sequence 1-35) is used the as an antigen, specifically sequence SETSRTAFGGRRAVPPNNSNAAEDDLPTVELQGVV, to produce specific antibodies against the stable double β-strands structure, effectively enhancing antibody sensitivity. More preferably, cysteine is added to the carboxyl end of the above partial sequence, and sensitivity experiments with the improved antibodies demonstrate that antibodies produced using this method can detect more than 80% of native free FXIIIAP.

The capture and detection antibodies are extracted from IgG of animals immunized with FXIIIAP. The detection antibody, after binding with biotin, is used in conjunction with Streptavidine alkaline phosphatase for enhanced sensitivity.

The method and the detection kit provide by the present invention can be used for quantitative analysis. As thrombus formation consumes FXIIIA, the A subunit of Coagulation Factor XIII (FXIIIA), FXIIIA also decreases in the blood. Although FXIIIA does not have a definitive diagnostic significance like FXIIIAP and D-dimer, the decrease thereof can indicate extent and severity of thrombus formation, and suggests more significant thrombus formation and severe condition.

### Embodiment 4

The present invention provides a method for detecting thrombosis and coagulation-related diseases, comprising the following steps:
S1: Producing specific antibodies using FXIIIAP as an antigen;
S2: Binding the obtained specific antibodies to free FXIIIAP in a test sample to form a complex;
S3: Detecting the formed complex to determine activation of FXIII and diagnose thrombosis or cardiovascular-related diseases.

Optionally, the test sample may be blood or plasma.

Optionally, specific antibodies are produced using a partial sequence of FXIIIAP as an antigen; the partial sequence of FXIIIAP includes sequence SETSRTAFGGRRAVPPNNSNAAEDDLPTVELQGVV or RTAFGGRRAVPPNN.

Wherein, the test sample is blood or plasma.

For better understanding of the above technical solutions, the exemplary embodiments of the present invention are described in more detail below with reference to the accompanying drawings. It should be understood that the present invention can be implemented in various forms and is not limited to the embodiments described herein. Instead, the embodiments are provided for clearer and more thorough understanding of the present invention and to fully convey the scope of the invention to the technical personnel in the field.

### Example 1

For preparation of the antibodies in the detection kit, the FXIIIAP partial sequence (SETSRTAFGGRRAVPPNNSNAAEDDLPTVELQGVV) was used as an antigen, and cysteine was added at the amino terminus to bind to a carrier protein, resulting in specific antibodies. The method for producing the specific antibodies could be common antigen-induced antibody generation techniques.

### Example 2

For preparation of the antibodies in the detection kit, the FXIIIAP complete sequence (SETSRTAFGGRRAVPPNNSNAAEDDLPTVELQGVVPR) was used as an antigen, and cysteine was added at the amino terminus to bind to a carrier protein, resulting in specific antibodies. The method for producing the specific antibodies could be common antigen-induced antibody generation techniques.

### Example 3

The FXIIIAP detection kit included: washing solution, sample diluent, chromogenic substrate, enzyme-labeled plate coated with capture antibodies, and detection antibodies.

The specific antibodies from Example 1 were used as capture antibodies, and the complete sequence of FXIIIAP from Example 2 was used as detection antibodies.

The method for preparing detection antibodies was as follows: the standard complete sequence FXIIIAP (SETSRTAFGGRRAVPPNNSNAAEDDLPTVELQGVVPR) was bound to a carrier protein, mice were immunized, and the resulting antibodies were purified with G protein. The detection antibodies must be conjugated with biotin. In the detection process, the detection antibodies were necessary to be combined with Streptavidine alkaline phosphatase to increase sensitivity.

The use of the above detection kit included at least the following steps:
Coating each well of the ELISA enzyme-labeled plate with 100 μl of capture antibody;
Blocking each well with bovine serum albumin to prevent non-specific binding;
After the capture antibody binds to the patients' plasma, adding biotinylated detection antibody (Detection Antibody), followed by adding Streptavidine alkaline phosphatase, and then introducing the chromogenic substrate;
Analyzing the results by using standard ELISA analysis equipment.

### Example 4

A combined detection kit comprising the FXIIIAP detection kit of Example 3 and a D-dimer quantitative detection kit, wherein the D-dimer quantitative detection kit can be a conventional detection kit.

The combined detection kit in this example could simultaneously detect FXIIIAP and D-dimer in patients' blood or plasma samples, significantly enhancing the detection rate of thrombosis and coagulation-related diseases.

To validate the feasibility of the detection kit and the related detection method in the present invention, initial clinical data from testing the detection kits were given. The following results are for the early diagnosis of ischemic stroke.

### Experiment 1: Sample Testing

122 samples from patients with early ischemic stroke were tested.

### 1. Statistical Method

Plasma samples were collected on the day symptoms appeared, and were labeled as Day 1, and subsequent samples were set as Day 2, Day 3, and Day 4 according to the collection time. Testing was conducted by using the combined detection kit of Example 4. In this test, the D-dimer level exceeding 500 ng/ml was considered as positive, and the FXIIIAP level more than 7 ng/ml was tentatively considered as positive.

### 2. Sensitivity Comparison

Sensitivity, also known as true positive rate, refers to the proportion of samples that are actually positive and are correctly identified as positive. In this experiment, the patients with actual ischemic stroke were identified based on FXIIIAP and/or D-dimer test results. The sensitivity was calculated as the ratio of true positives to the sum of true positives and false negatives (true positives but identified as negatives).

### Test Results:

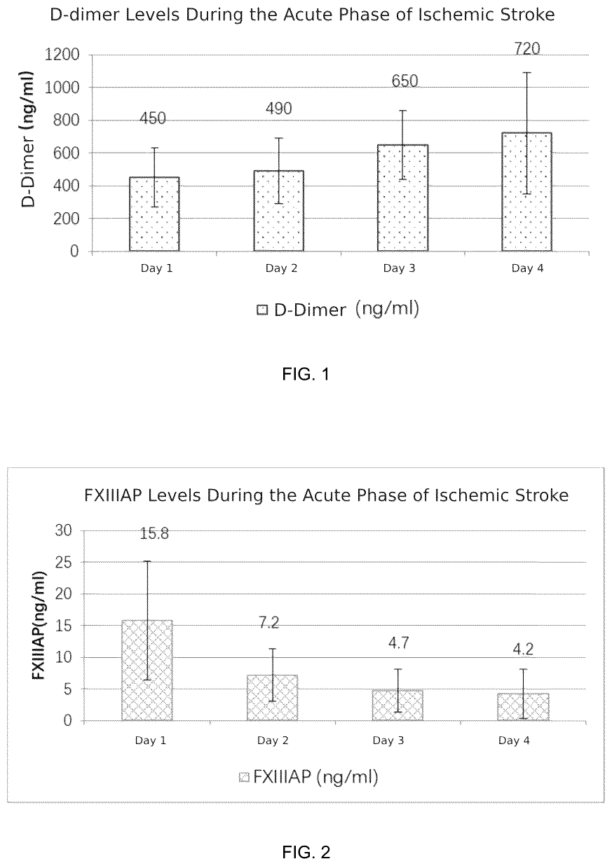

Refer to FIG. 1, which showed the test results of ischemic stroke patient samples using the traditional marker D-dimer as a control for the present invention. FIG. 1 indicated that among the 122 patient samples, the D-dimer levels significantly increased on Day 3, exceeding 500 ng/ml, while the average concentration on Day 1 was lower than 500 ng/ml.

Refer to FIG. 2, which showed the test results of ischemic stroke patient samples using FXIIIAP as the marker. FIG. 2 indicated that the FXIIIAP concentration changes were opposite to D-dimer: high concentration of 15.8 ng/ml appeared on Day 1, decreased to 7.2 ng/ml on Day 2, and reduced to 4 ng/ml on Days 3 and 4.

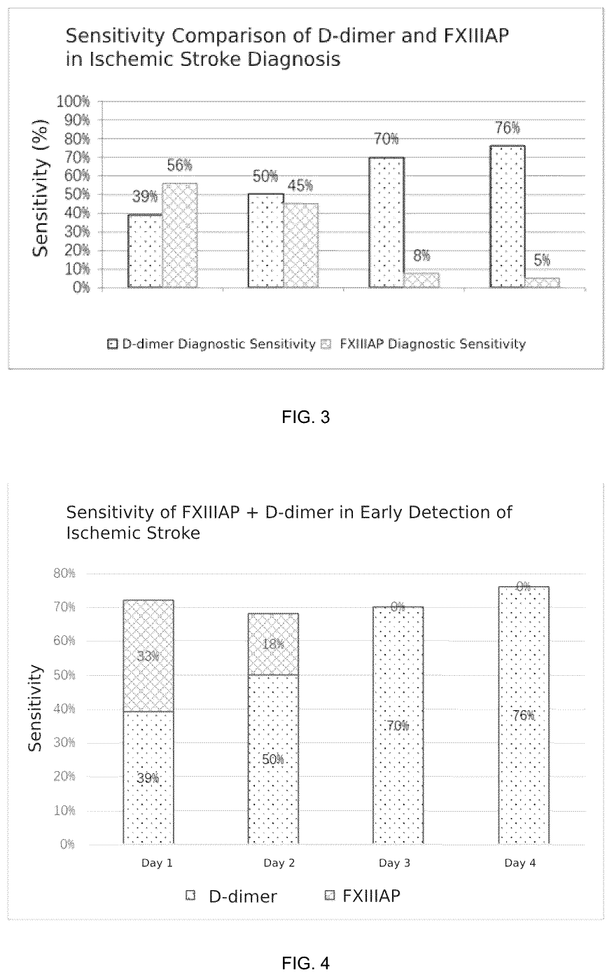

FIG. 3 was a comparison diagram of the sensitivities of marker FXIIIAP and traditional marker D-dimer used for early ischemic stroke patient samples. FIG. 3 showed that on Day 1, the sensitivity of FXIIIAP was higher than D-dimer, similar on Day 2; however, the sensitivity of D-dimer was higher than FXIIIAP on Days 3 and 4. Combining both markers, i.e., the D-dimer level of more than 500 ng/ml or the FXIIIAP level of more than 7 ng/ml was set as positive, the sensitivity for Days 1 and 2 increased to 72% and 68%, respectively. This is a significant improvement compared to using only D-dimer for diagnosis (39% on Day 1, 50% on Day 2).

FIG. 4 illustrated the sensitivity of the combination of FXIIIAP and D-dimer markers for early ischemic stroke patient samples. The combination of FXIIIAP and D-dimer showed excellent diagnostic performance for early ischemic stroke.

### Experiment 2

### Testing Samples

27 plasma samples (collecting on the day symptoms appear) from patients with early hemorrhagic stroke were tested using a combination of the FXIIIAP detection kit and the D-dimer quantitative detection kit. The results showed that the combined sensitivity of detecting FXIIIAP and D-dimer reached 100%, without missed diagnoses. In contrast, testing with only D-dimer detection kit showed a missed diagnosis rate of 22%. Therefore, the combined detection kit proposed in the present invention is beneficial to reduce the missed diagnosis rate for hemorrhagic stroke using D-dimer.

### Experiment 3

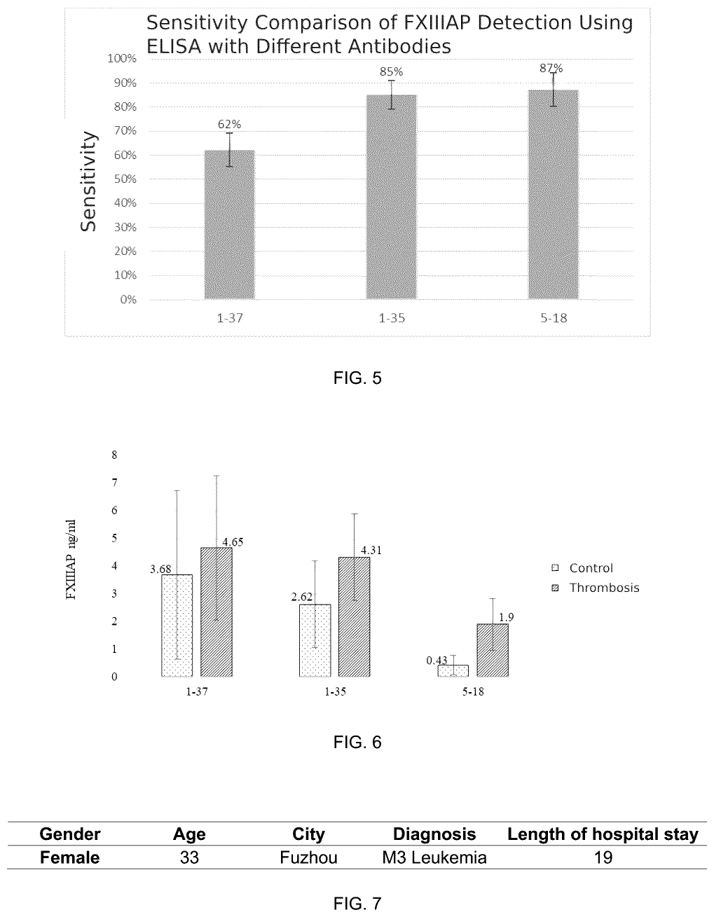

Refer to FIG. 5, which compared the sensitivity of antibodies described in Embodiment 3-1-1 (original antibody, labeled as 1-37), Embodiment 3-1-2 (improved antibody 1, labeled as 1-35), and Embodiment 3-1-4 (improved antibody 2, labeled as 5-18) for FXIIIAP detection using ELISA. Data from FIG. 5 revealed that FXIIIAP could not detected in approximate 40% of plasma samples in vitro tests (using normal plasma) by using the detection kit containing the original antibody. The poor sensitivity also led to suboptimal results in clinical tests. This may be due to the inability of the obtained specific capture antibodies to efficiently capture FXIIIAP in plasma.

Conversely, for the same plasma samples, FXIIIAP was detected in 85% and 87% of the plasma samples in vitro test using the detection kit containing either improved antibody 1 or improved antibody 2. Laboratory results demonstrated that improved antibodies 1 or 2 as capture antibodies better identified FXIIIAP in vivo, without cross-reacting with FXIII, thereby accurately reflecting the real levels of FXIIIAP in test samples.

Therefore, due to the improvement in capture antibodies, the sensitivity for early detection of FXIIIAP in patients increased by about 30%. Furthermore, due to the rapid degradation characteristics of FXIIIAP, the aforementioned detection kits achieved better detection rates for early-stage coagulation-related diseases or thrombosis. In contrast, there exists a missed diagnosis rate for later-stage coagulation-related diseases or thrombosis.

### Experiment 4

### 1. Testing Samples

Plasma samples from 40 normal individuals were selected as the control group, and plasma samples from 40 hospitalized leukemia patients with thrombosis were used the test group. Samples were collected on the day symptoms appeared (the day of diagnosis) and tested. Data were the average values or average values±standard deviation of 40 parallel experiments.

### 2. FXIIIAP Detection Kit

FXIIIAP detection kit Included washing solution, sample diluent, chromogenic substrate, enzyme-labeled plate coated with capture antibody, and detection antibody.

Wherein, the capture antibodies were as described in Embodiment 3-1-1 (original antibody, SETSRTAFGGRRAVPPNNSNAAEDDLPTVELQGVVPR), Embodiment 3-1-2 (improved antibody 1, SETSRTAFGGRRAVPPNNSNAAEDDLPTVELQGVV), and Embodiment 3-1-4 (improved antibody 2, RTAFGGRRAVPPNN).

The detection antibody was the complete sequence of FXIIIAP (SETSRTAFGGRRAVPPNNSNAAEDDLPTVELQGVVPR). The method for preparing the detection antibodies was as follows: the standard complete sequence FXIIIAP (SETSRTAFGGRRAVPPNNSNAAEDDLPTVELQGVVPR) was bound to a carrier protein, mice were immunized, and the resulting antibodies are purified with G protein. The detection antibodies must be conjugated with biotin.

The use of the detection kit included at least the following steps:
Coating each well of the ELISA enzyme-labeled plate with 100 μl of capture antibody;
Blocking each well with bovine serum albumin to prevent non-specific binding;
After the capture antibody binds to the patients' plasma, adding biotinylated detection antibody (Detection Antibody), followed by adding Streptavidine alkaline phosphatase, and then introducing the chromogenic substrate;
Analyzing the results by using standard ELISA analysis equipment.

### 3. Specificity Comparison

Specificity refers to effective detection of free-state FXIIIAP, without binding to unactivated FXIIIAP (bound-state FXIIIAP). Non-specificity refers to interaction of bound-state FXIIIAP on unactivated FXIII with antibodies, thereby affecting experimental values.

It is important to note that due to the specificity test, blood samples from thrombotic leukemia patients with lower FXIIIAP levels were used, and samples from stroke patients were not tested (FXIIIAP levels in stroke patients are very high [>7 ng/ml], unsuitable for specificity comparison). Unlike stroke patients (wherein the opening of the blood-brain barrier allows a large amount of tissue factors into the blood, causing widespread thrombosis and a rapid increase in FXIIIAP), thrombotic leukemia patients have lower and less variable FXIIIAP concentrations in localized thrombosis, thus requiring higher specificity in antibodies for a more direct and valid reflection of test data.

### 4. Test Results:

Refer to Table 1 for specificity comparison data of FXIIIAP detection using ELISA with antibodies described in Embodiment 3-1-1 (original antibody, labeled as 1-37), Embodiment 3-1-2 (improved antibody 1, labeled as 1-35), and Embodiment 3-1-4 (improved antibody 2, labeled as 5-18).

TABLE 1

| | Normal Control group | | Thrombosis experimental group | |
|---|---|---|---|---|
| | Average | Standard | Average | Standard |
| | ng/ml | Deviation (SD) | ng/ml | Deviation (SD) |
| 1-37 | 3.68 | 3.05 | 4.65 | 2.61 |
| 1-35 | 2.62 | 1.57 | 4.31 | 1.55 |
| 5-18 | 0.43 | 0.36 | 1.90 | 0.94 |

Refer to FIG. 6 for specificity comparison of FXIIIAP using ELISA with antibodies as described in Embodiment 3-1-1 (original antibody, labeled as 1-37), Embodiment 3-1-2 (improved antibody 1, labeled as 1-35), and Embodiment 3-1-4 (improved antibody 2, labeled as 5-18).

From the data in Table 1 and FIG. 6, it was evident that all antibodies could detect FXIIIAP in the samples. However, Improved Antibody 1 (labeled as 1-35) showed a lower concentration of FXIIIAP (2.62 ng/ml) in the normal control group than the original antibody. Improved Antibody 2 (labeled as 5-18) detected even lower FXIIIAP concentrations (0.43 ng/ml). Theoretically, FXIIIAP is only detectable when activated, hence it is present at very low values in normal human bodies (currently, there are no precise FXIIIAP detection methods available globally, apart from the antibodies and detection kits described in the present invention). Comparing the control group data and the experimental group data, there was a statistically significant difference (P<0.01) between Improved Antibody 2 in the control group and the experimental group. Similarly, Improved Antibody 1 showed a statistically significant difference (P<0.05), while the original antibody (complete sequence 1-37) showed no significant difference (P>0.05) between the control group and the experimental group. This indicated that for thrombosis detection, the improved antibodies (1 and 2) in the present invention can detect FXIIIAP levels closer to the actual values, making them being suitable for real-time and effective detection of thrombosis in hospitalized patients.

### Embodiment 5

For preparation of antibodies in the detection kit, the partial sequence of FXIIIAP (RTAFGGRRAVPPNN) was used as an antigen, and cysteine was added at amino terminus thereof to bind to a carrier protein, resulting in specific antibodies. The method for producing the specific antibodies can be the conventional antigen-induced antibody production techniques.

### Embodiment 6

The FXIIIAP detection kit (dry-type immunofluorescence quantitative method) included a PVC base plate, a fluorescence conjugate pad, a nitrocellulose membrane, a sample pad, and an absorbent pad, wherein the fluorescence conjugate pad, the nitrocellulose membrane, the sample pad, and the absorbent pad were all fixed on the PVC base plate. The fluorescence conjugate pad contains detection antibodies labeled with fluorescent microspheres, and the nitrocellulose membrane sequentially involves test lines composed of capture antibodies and quality control lines composed of rabbit anti-mouse IgG antibodies.

Specific antibodies from Embodiment 5 were used as both capture antibodies and detection antibodies.

The preparation method for the detection antibody (detection antibody) was as follows: The partial sequence of FXIIIAP (improved antibody 2, RTAFGGRRAVPPNN) was combined with a carrier protein, mice was immunized, and the obtained antibodies were purified using G protein.

The basic principle the aforementioned of fluorescent immunochromatographic detection kit is double-antibody sandwich method, wherein analyte in the sample is able to bind to the detection antibodies labeled with fluorescent microspheres (improved antibody 2-fluorescent microspheres) dispersed in the conjugate pad, thereby forming complexes. The complexes migrate forward along the reaction membrane under chromatography, and are captured by the corresponding capture antibodies (improved antibody 2) at the test line on the reaction membrane. The more analytes in the sample, the more complexes formed, leading to more accumulation of complexes at the test line and a more pronounced color or fluorescence signal, which indicates the quantity of the captured analytes. Unreacted detection antibodies marked with fluorescent microspheres migrate to the quality control line and then bind to the rabbit anti-mouse IgG antibodies, indicating validity. Based on standard curves, the concentration of FXIIIAP in the test samples can be calculated.

The use of the aforementioned kit included the following steps at least:

The test sample was added to the sample well of the test strip. After a 15-minute reaction, a dry immunofluorescence analyzer was used to read the fluorescence signals at the T (Test) and C (Control) lines on the nitrocellulose membrane to determine the concentration of FXIIIAP in the sample.

To verify the feasibility of the biomarkers, detection kits, and detection methods described in the present invention, initial clinical data from applications of the detection kits were presented below. The results pertained to early diagnosis tests in cases of coagulation abnormal thrombosis caused by M3 leukemia, thrombotic thrombocytopeniaurpura, and deep vein thrombosis, and ischemic stroke.

### Experiment 5

Real-time monitoring of coagulation abnormalities and thrombosis caused by M3 leukemia was conducted using the FXIIIAP detection kit of Embodiment 6 and D-dimer quantitative detection kit, wherein the FXIIIAP detection kit was used to monitor changes in FXIIIAP, and the D-dimer quantitative detection kit was for D-dimer changes. Patients A and B, suffering from coagulation abnormalities and thrombosis due to M3 leukemia, were monitored for various biochemical indicators (including FXIIIAP and D-dimer) from admission to 18-20 days of treatment.

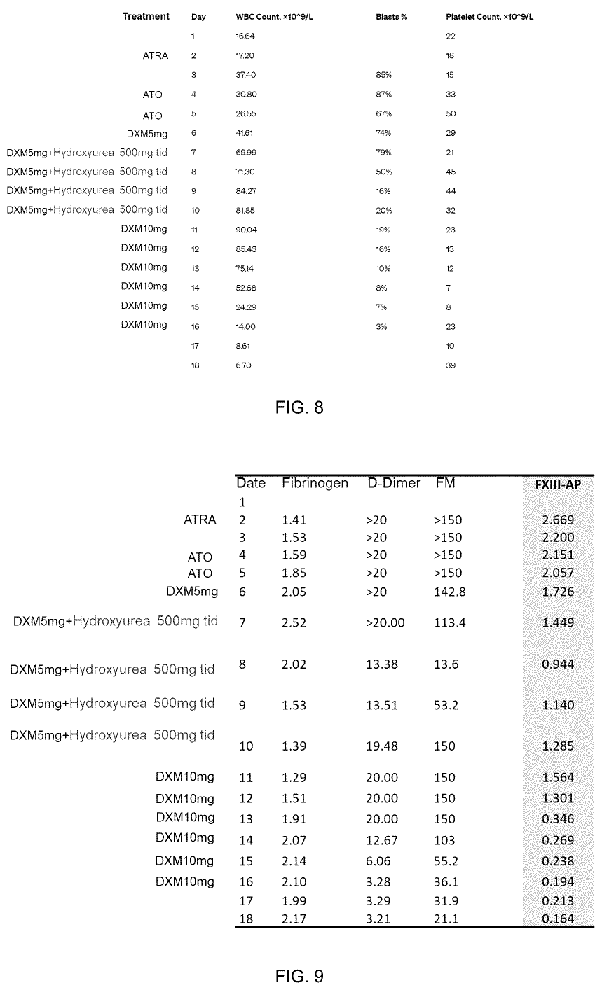

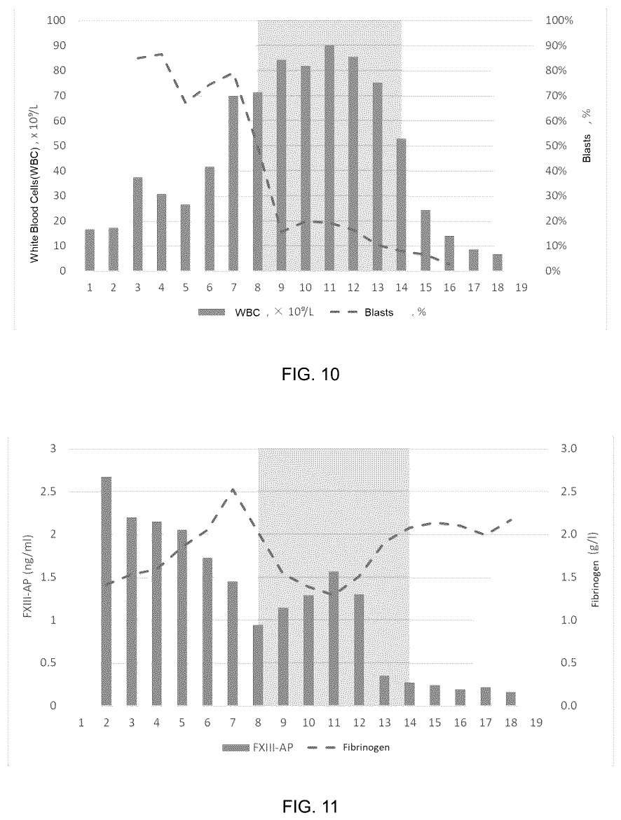

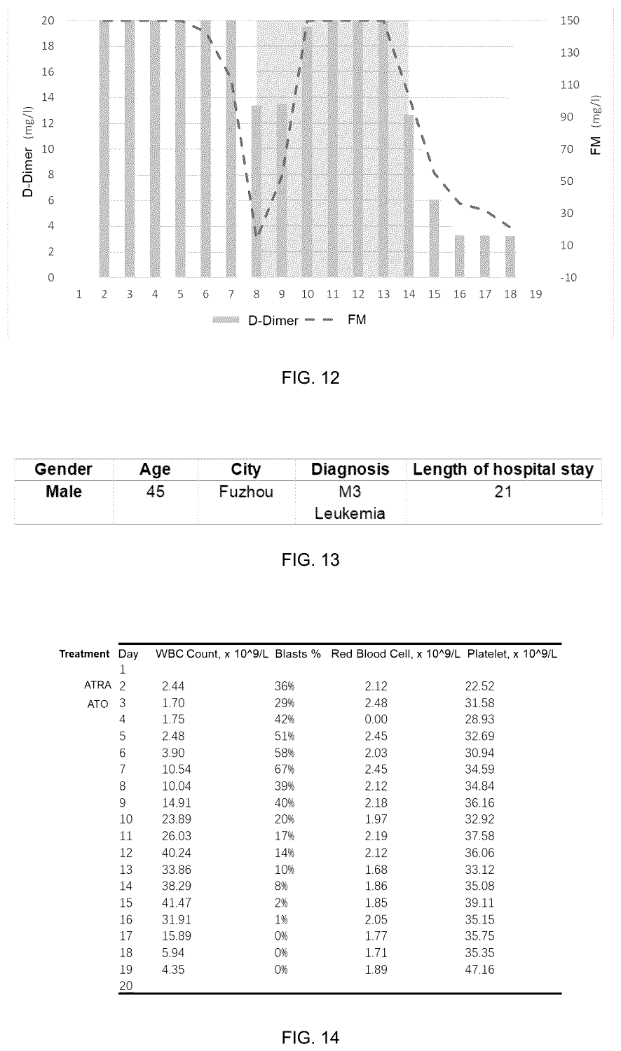

Refer to FIGS. 7-12. FIG. 7 showed basic information of Patient A. FIGS. 8 and 9 showed thrombosis monitoring data of Patient A. FIG. 10 showed changes in white blood cells and promyelocyte ratio for Patient A. FIG. 11 showed changes of FXIIIAP and fibrinogen in Patient A. FIG. 12 showed changes of D-dimer and fibrin monomer in Patient A.

FIGS. 8 and 10 indicated that during the treatment, the decrease of promyelocyte ratio and the increase of white blood cells led to the release of procoagulant substances, which caused microthrombi formation and induced differentiation syndrome [symptoms of differentiation syndrome appearing on days 8-14 (corresponding to the gray highlighted background in FIGS. 10-12), suggesting thrombosis formation].

FIGS. 9 and 11 indicated that during the period of differentiation syndrome, the FXIIIAP level increased, and the change trend thereof was consistent with that of white blood cell levels. The change trend of fibrinogen was consistent with that of FXIIIAP. Thrombus formation could lead to fibrinogen consumption, thereby supporting thrombus formation in vivo, further confirming the accuracy of FXIIIAP monitoring. After initial treatment, the FXIIIAP levels decreased, which suggested an improvement in thrombus and coagulation abnormalities.

FIGS. 9 and 12 revealed that the change trends of D-dimer (a thrombus degradation product, indicating thrombolysis) and fibrin monomer (FM, indicating a hypercoagulable state) were consistent with that of FXIIIAP. However, as both markers (D-dimer and fibrin monomer) were not direct indicators of thrombus formation and exceeded maximum detection values during early onset and differentiation syndrome periods, they were challenging to be used for thrombus in vivo monitoring.

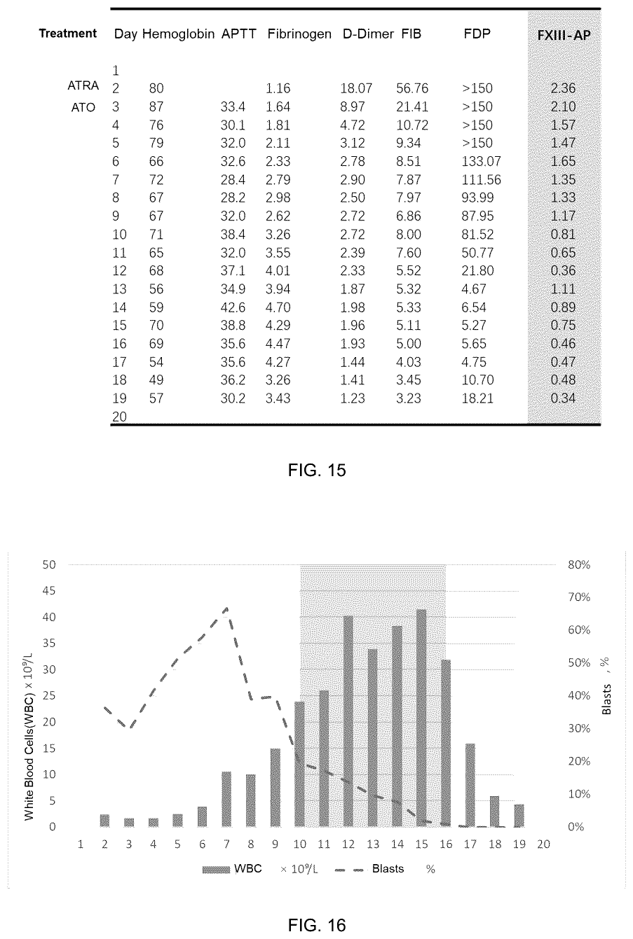

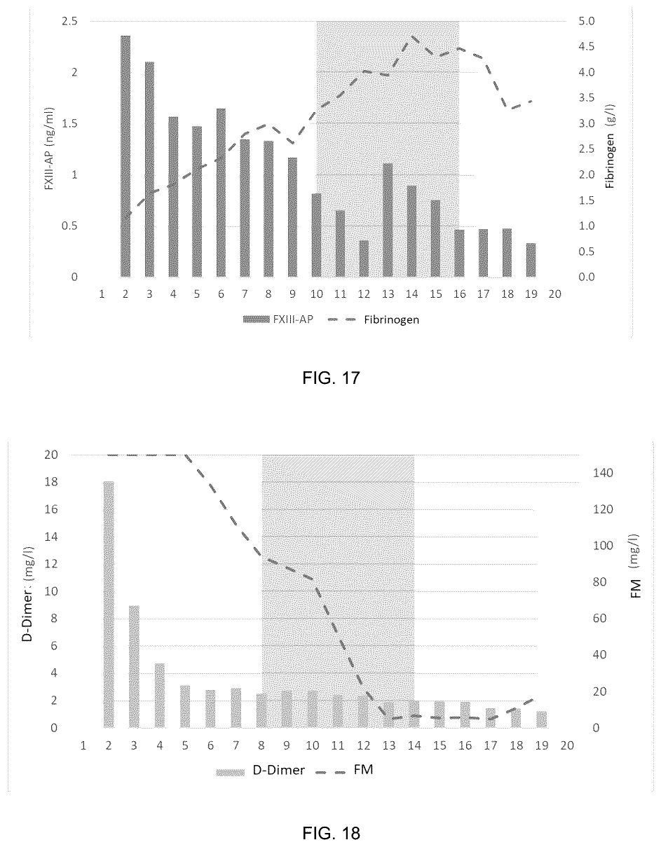

Refer to FIGS. 13-18. FIG. 13 showed basic information of Patient B. FIGS. 14 and 15 showed thrombosis monitoring data of Patient B. FIG. 16 shows monitoring changes in white blood cells and promyelocytes ratio for Patient B. FIG. 17 showed monitoring changes of FXIIIAP and fibrinogen in Patient B. FIG. 18 showed monitoring changes of D-dimer and fibrin monomer in Patient B.

Monitoring coagulation abnormalities and thrombotic risks was crucial in treatment of M3 leukemia, as indicated in the data from FIGS. 14-18. After treatment with retinoic acid, a rapid decrease in FXIIIAP levels was observed, indicating a good response to the medication and a good control of thrombosis and coagulation abnormalities, thus there was no need to increase chemotherapy drugs. FIG. 18 showed that after a decline, D-dimer and fibrin monomer levels quickly reached a plateau, thereby losing the effectiveness thereof in thrombus monitoring.

This indicated that monitoring thrombosis in vivo using the improved antibodies for FXIIIAP detection in the present invention can accurately control medication timing, reducing or even avoiding unnecessary chemotherapy, lowering infection risks, reducing hospitalization costs, and increasing cure rates.

### Experiment 6

The combination of FXIIIAP detection kit of Embodiment 6 and D-dimer quantitative detection kit combination were used for real-time monitoring of thrombotic thrombocytopenia purpura and deep vein thrombosis, wherein the FXIIIAP detection kit was applied to monitor FXIIIAP changes and the D-dimer quantitative detection kit was for D-dimer changes. Patient C, suffering from thrombotic thrombocytopeniaurpura and deep vein thrombosis, was monitored for various biochemical indicators (including FXIIIAP and D-dimer) at admission, after one week of treatment to condition improvement, and upon disease relapse.

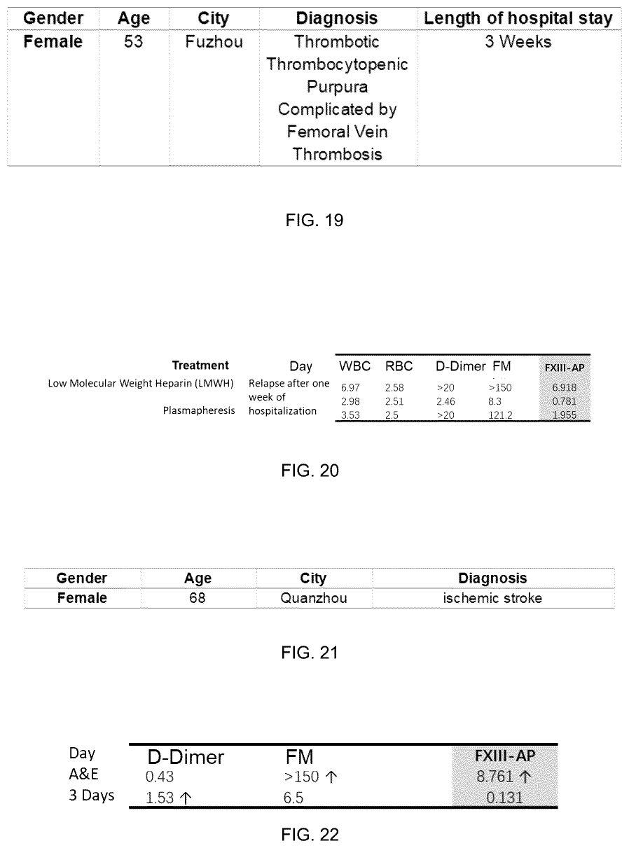

Refer to FIGS. 19 and 20. FIG. 19 showed basic information of Patient C. FIG. 20 presented thrombosis monitoring data for Patient C. FIG. 20 indicated that at the peak of thrombus formation at admission, the FXIIIAP level was high and rapidly decreased after the treatment. Upon relapse and readmission, the FXIIIAP level rose again. This showed that the FXIIIAP level was correlated with the patients' conditions, proving the effectiveness thereof as a biomarker for thrombosis in vivo monitoring.

### Experiment 7

The combination of FXIIIAP detection kit of Embodiment 6 and D-dimer quantitative detection kit combination were used for real-time monitoring of ischemic stroke, wherein the FXIIIAP detection kit was used to monitor FXIIIAP changes and the D-dimer quantitative detection kit was for D-dimer changes. Patient D, suffering from ischemic stroke, was monitored for various biochemical indicators (including FXIIIAP and D-dimer) from emergency hospital admission and throughout treatment.

Refer to FIGS. 21 and 22. FIG. 21 showed basic information of Patient D. FIG. 22 presented thrombosis monitoring data for Patient D. FIG. 22 indicated that upon hospital admission, the FXIIIAP level was high while the D-dimer level was still within the normal range (when D-dimer testing was used for detecting fibrinolysis products, there was a delay, thus traditional D-dimer testing methods had a risk of missed diagnosis). After the patient's condition was stable after treatment, the FXIIIAP level decreased rapidly and the D-dimer level increased.

In conclusion, the data from the above experiments indicated that the combination of FXIIIAP and D-dimer biomarkers proposed in the present invention was able to effectively reduce the missed diagnosis rate and provided feedback on the formation and the stabilization of thrombosis in vivo. The antibodies of the present invention was able to be applied in various existing methodologies, such as enzyme-linked immunosorbent assay (ELISA) and dry immunofluorescence, in order to effectively detect thrombotic or coagulation-related diseases.

Importantly, it should be noted: the embodiments described above are intended to illustrate the technical solutions of the present invention and are not limitations thereof. Despite detailed descriptions of the invention through the aforementioned embodiments, it should be understood by those skilled in the art that modifications to the technical solutions recorded in the aforementioned embodiments, or partial or complete equivalent replacements of some or all of the technical features thereof, do not depart from the essence of the technical solutions of the embodiments of the present invention, which was covered by the present invention.

1. A biomarker for detecting thrombotic or coagulation-related disorders, characterized by comprising a blood coagulation factor 13 activation peptide or/and a blood coagulation factor 13 A subunit.

2. The biomarker for detecting thrombotic or coagulation-related disorders according to claim 1, characterized by comprising a combination of the blood coagulation factor 13 activation peptide and D-dimer.

3. The biomarker for detecting thrombotic or coagulation-related disorders according to claim 1, characterized in that the coagulation-related diseases include stroke, cerebral venous thrombosis, renal venous thrombosis, venous thrombosis or myocardial infarction.

4. An application of biomarkers as described in claim 1 in products for detecting thrombotic or coagulation-related disorders and/or assessing therapeutic effects.

5. The application of biomarkers as described in claim 4 in products for detecting thrombotic or coagulation-related disorders and/or assessing therapeutic effects, characterized in that the products include a reagent kit or a reagent.

6. A detection kit for detecting thrombosis or coagulation-related diseases, characterized by comprising a capture antibody and/or a detection antibody, which is a specific antibody prepared by using the biomarker described in claim 1 as an antigen.

7. The detection kit for detecting thrombotic or coagulation-related disorders according to claim 6, characterized in that the detection kit is a double-antibody sandwich ELISA detection kit and/or a dry-type immunofluorescence detection kit, including a capture antibody and a detection antibody.

8. The detection kit for detecting thrombotic or coagulation-related disorders according to claim 6, characterized in that the capture antibody and the detection antibody are prepared by using complete sequence or partial sequence of FXIIIAP as an antigen.

9. The detection kit for detecting thrombotic or coagulation-related disorders according to claim 8, characterized in that the partial sequence includes the following sequence or a fragment comprising the following sequence: SETSRTAFGGRRAVPPNNSNAAEDDLPTVELQGVV or RTAFGGRRAVPPNN.

10. The detection kit for detecting thrombotic or coagulation-related disorders according to claim 7, characterized in that the capture antibody and the detection antibody are extracted from a FXIIIAP immunized test animal.

11. The biomarker for detecting thrombotic or coagulation-related disorders according to claim 2, characterized in that the coagulation-related diseases include stroke, cerebral venous thrombosis, renal venous thrombosis, venous thrombosis or myocardial infarction.

12. The detection kit for detecting thrombotic or coagulation-related disorders according to claim 6, characterized in that the biomarkers mentioned include a combination of blood coagulation factor 13 activation peptide and D-dimer.

13. The detection kit for detecting thrombotic or coagulation-related disorders according to claim 12, characterized in that the detection kit is a double-antibody sandwich ELISA detection kit and/or a dry-type immunofluorescence detection kit, including a capture antibody and a detection antibody.

14. The detection kit for detecting thrombotic or coagulation-related disorders according to claim 12, characterized in that the capture antibody and the detection antibody are prepared by using complete sequence or partial sequence of FXIIIAP as an antigen.

15. The detection kit for detecting thrombotic or coagulation-related disorders according to claim 14, characterized in that the partial sequence includes the following sequence or a fragment comprising the following sequence: SETSRTAFGGRRAVPPNNSNAAEDDLPTVELQGVV or RTAFGGRRAVPPNN.

16. The detection kit for detecting thrombotic or coagulation-related disorders according to claim 13, characterized in that the capture antibody and the detection antibody are extracted from a FXIIIAP immunized test animal.
